(19) United States
(12) Patent Application Publication
(10) Pub. No.: US 2026/0316073 A1
(43) Pub. Date: Oct. 15, 2026

(54) MICROFLUIDIC NUCLEIC ACID EXTRACTION

(71) Applicant: UNIVERSITY COLLEGE DUBLIN, Dublin (IE)

(72) Inventors: Akshaya JAGANNATH, Dublin (IE); Yinghao LI, Dublin (IE); Hengji CONG, Dublin (IE); Gabriel GONZALEZ, Dublin (IE); Jaythoon HASSAN, Dublin (IE); Wenxin WANG, Dublin (IE); Michael GILCHRIST, Dublin (IE); Nan ZHANG, Dublin (IE)

(21) Appl. No.: 19/481,139

(22) PCT Filed: Apr. 29, 2024
(86) PCT No.: PCT/EP2024/061746

§ 371 Date: Nov. 3, 2025

(30) Foreign Application Priority Data
May 5, 2023 (GB) 2306715.0

(51) Int. Cl. B01L 3/00; C12Q 1/6806; C12Q 1/6844

(52) U.S. Cl. CPC B01L 3/502715; C12Q 1/6806; C12Q 1/6844; B01L 2200/027; B01L 2200/028; B01L 2200/0631; B01L 2200/10; B01L 2200/16; B01L 2300/0819; B01L 2300/0861; B01L 2400/043

(57) ABSTRACT

A microfluidic nucleic acid extraction chip (1), the chip (1) comprising a membrane-containing chamber (6) bonded between a first layer (2) and a second layer (3), wherein the first layer (2) comprises a sample inlet port (4) in fluid communication with the membrane-containing chamber (6) by a first microchannel (8), and a wash buffer inlet port (10) and an elution buffer waste collection port (12) in fluid communication with the membrane-containing chamber (6) by a second microchannel (14); wherein the second layer (3) comprises an elution buffer inlet port (5) in fluid communication with an eluate collection port (7) adapted to deliver extracted nucleic acid to a nucleic acid amplification module via a third microchannel (9); and wherein the membrane-containing chamber (6) is in fluid communication with the second layer (3) through an extraction microchannel (11); and the membrane-containing chamber (6) contains a silica membrane modified with a hyperbranched, cationic group.

### FIELD OF THE INVENTION

The invention relates to extracting nucleic acid efficiently on a microfluidic chip. Specifically, the invention relates to a microfluidic method capable of nucleic acid extraction using a modified silica membrane in a rapid and instrument-free manner without the presence of amplification inhibitors.

### BACKGROUND TO THE INVENTION

Silica is the matrix generally used for solid phase extraction (SPE) due to its historical use of purifying DNA from agarose gels. The process involves using a lysis buffer containing detergent to dissolve membrane lipids and proteins, which causes the release of cell contents through pores. The DNA is then exposed to high salt and low pH conditions, leading to the disruption of non-covalent bonds in the phosphate backbone. The surplus of negative charges on the DNA allows it to bind to the positively charged surface of the silica through electrostatic interaction. However, other biomolecules in the lysed cell can non-specifically bind to these surfaces and can be removed through selective washing with cleaving enzymes, while ethanol can maintain the bound DNA to the surface. When pH is increased, the negative charge density on the membrane surface results in greater electrostatic repulsion between the DNA and silica surface causing elution. The wide adaptation of silica surfaces is due to its stability, biocompatibility and easily modified properties in solid phase extraction. However, a common problem is the carry-over of residual chaotropic salts and ethanol into the amplification reaction, which can inhibit polymerase amplification.

Although silica spin column methods are used in laboratory settings which have good resources and equipment, they present a myriad of challenges in point-of-care technology integration. These methods are labour intensive (>20 steps, multiple reagents, multiple wash/centrifuging cycles), time-consuming (1-2 hours on average) and are restricted to certain specimens or types of nucleic acids (separate products for extraction from saliva, blood, etc.). This greatly limits their potential in one-pot systems. To overcome these limitations, microfluidic-based point-of-care devices have been developed that can accommodate on-chip SPE using versatile silica structures: silicon-based micropillars, silicon nanowires, rotating microfluidic systems using silicon beads, and monodisperse-porous silica microspheres on the microfluidic chip.

Recent advances in on-chip nucleic acid extraction from blood include enhanced solid-phase extraction by combining ultrasonic cell lysis with silica membrane-based SPE for rapid sample preparation from serum samples. Another method involves surface modification of a plastic substrate by introducing additional positive charges to facilitate DNA capture via electrostatic binding or complexing with the cationic polymer (Choi, Y., et al., All-in-one pumpless portable genetic analysis microsystem for rapid naked-eye detection. Sensors and Actuators B: Chemical, 2021. 344: p. 130307). For these systems, the microchip requires ~30 min of incubation to capture 90% DNA from cell lysate.

Alternatively, another method using polymer monolith columns can be impregnated with silica particles to form a silica-polymer composite to directly extract nucleic acids from blood (Mahalanabis, M., et al., Cell lysis and DNA extraction of gram-positive and gram-negative bacteria from whole blood in a disposable microfluidic chip. Lab on a Chip, 2009. 9 (19): p. 2811-2817). However, the non-specific binding to any negatively charged molecules present in the sample is a major drawback of these systems.

Additionally, the detection of trace amounts of pathogens in clinical samples is challenging. For early detection, processing of whole blood or plasma in a microfluidic device without pathogen enrichment may not yield an amplifiable signal. Commercially adaptable sample extraction systems require ease of automation, integration with amplification and detection, minimizing manual steps and use of external instruments. Currently, these demands are hindered by high sample volume requirements (>1 mL), manual steps such as cartridge transfers, additional instrumentation, and increased duration (2-3 hours).

Microfluidic extraction is typically performed by filtering nucleic acids from clinical specimen using silica structures (to “trap” the desired nucleic acids) or using suitably coated magnetic beads. Once the nucleic acids are trapped, they are eluted by magnet-aided movement or centrifugation at high speeds. This presents several problems in large scale manufacturing of efficient biomarker detection devices:
(1) Complex fabrication of micro/nano scale silica features such as micropillars and nanowires, leading to high cost, reproducibility challenges, additional standardization and quality requirements.
(2) Multiple instrumentation required for unit operations (centrifugation, magnetic movement, transfer of samples) which results in manual error and low efficiency.
(3) Multi-component manufacturing adding to the time, cost and quality control requirements for mass manufacturing.
(4) Assembly and post-fabrication challenges (bonding) increase due to multiple components, material composition and scale of features.

It is the object of the subject application to overcome at least one of the above-mentioned problems.

### SUMMARY OF THE INVENTION

Integrating nucleic acid extraction in amplification-based point-of-care diagnostics will be a significant feature for next-generation point-of-care virus detection devices. However, extracting DNA efficiently on a microfluidic chip poses many technological and commercialization challenges, including manual steps, multiple instruments, pretreatment processes and use of organic solvents (ethanol, IPA) that inhibit detection and are not compatible with routine testing such as viral load monitoring of transplant patients. The invention described herein presents a microfluidic method capable of a two-step nucleic acid extraction from a low volume of a biological sample. In contrast to most previously reported or commercialized instruments, the microfluidic method built here lowers the number of steps (compared to manual extraction processes), is rapid (direct extraction from a biological sample is completed in 15-20 minutes) and has minimal on-chip components for ease of fabrication. Thus, this method can be a standalone extraction device or can be integrated with nucleic acid amplification platforms for direct on-chip detection of pathogens from a biological sample.

The core component of the method is a microfluidic (MF) chip. The MF chip comprises of two layers: a first (top) layer of a polymer substrate that includes an inlet channel for the biological sample and buffers, and an embedded UV-treated hyperbranched poly(β-amino ester) (HPAE)-modified (cationic) silica membrane; and a second (bottom) layer that includes a membrane extractant chamber and an outlet channel for collecting the purified extract. The membrane is used for detection of a target in a rapid and instrument-free manner without the presence of amplification inhibitors. HPAEs of varying branch ratios are synthesized, screened, and coated on the silica membrane and bonded between the two layers of polymer substrates. The method of the claimed invention can selectively extract nucleic acid from a biological sample with an efficiency of 94% and a lower limit viral load of 300 IU/mL in 20 minutes.

The extracted nucleic acid was used as the template for real-time LAMP-based detection of a viral load and was found to produce a fluorescent signal intensity that was comparable with commercially extracted templates. The claimed method can be integrated easily with a nucleic acid amplification system and used for routine rapid testing of virus or viral load in patient biological samples.

In one aspect, there is provided a method, a device, and a kit as set out in the appended claims.

In one aspect, there is provided a microfluidic nucleic acid extraction chip (1), the chip (1) comprising a membrane-containing chamber (6) bonded between a first layer (2) and a second layer (3), wherein the first layer (2) comprises a sample inlet port (4) in fluid communication with the membrane-containing chamber (6) by a first microchannel (8), and a wash buffer inlet port (10) and an elution buffer waste collection port (12) in fluid communication with the membrane-containing chamber (6) by a second microchannel (14); wherein the second layer (3) comprises an elution buffer inlet port (5) in fluid communication with an eluate collection port (7) adapted to deliver extracted nucleic acid to a nucleic acid amplification module via a third microchannel (9); and wherein the silica membrane-containing chamber (6) is in fluid communication with the second layer (3) through an extraction microchannel (11); and wherein the membrane-containing chamber (6) contains a silica membrane modified with a hyperbranched cationic polymer.

In one aspect, the hyperbranched cationic polymer has a cationic group selected from an amino ester, sulphonium, oxonium, phosphonium, imidazolium, pyrrolidinium, pyridinium, pipyridinium, morpholinium, and quinolinium.

In one aspect, the hyperbranched cationic polymer has a branch monomer to the linear monomer molar ratio of 0 to 0.1.

In one aspect, the hyperbranched cationic polymer has a molecular weight range of between about 5 kDa to about 65 kDa. Preferably, the hyperbranched cationic polymer has a molecular weight range of between about 5.3 kDa to about 64.0 kDa.

In one aspect, the hyperbranched cationic polymer is composed of a mixture of two or more of 1,4-butanediol diacrylate, 5-amino-1-pentanol, 3-Morpholinopropylamine, and pentaerythritol tetra-acrylate.

In one aspect, the hyperbranched cationic polymer has a chemical structure:

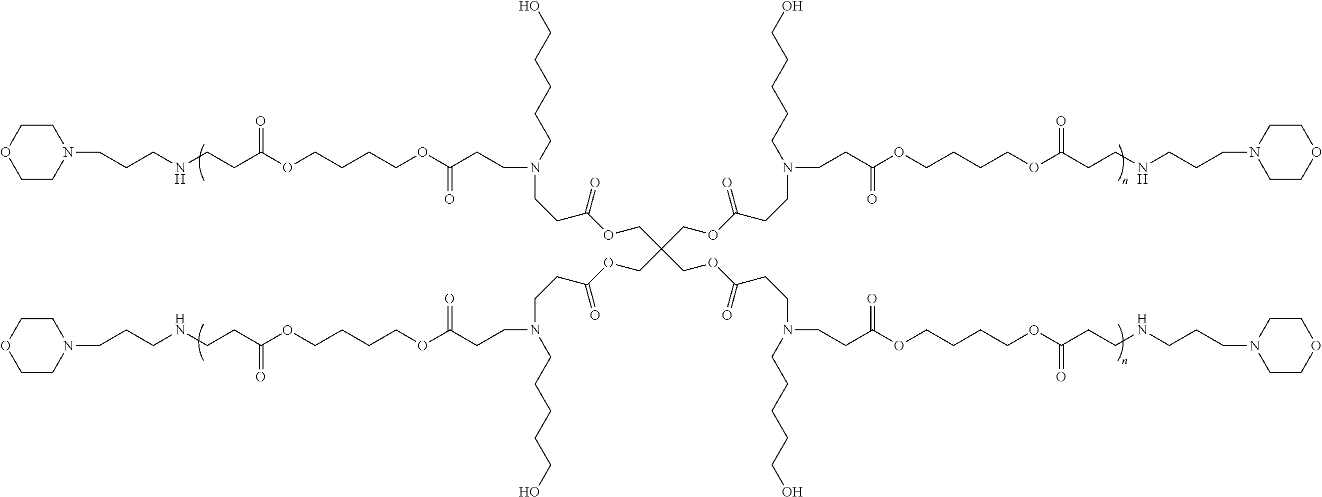

wherein n is the number of the repeating monomer unit between 1 and 500. These are referred to as E12 to E16 in the specification.

In one aspect, the hyperbranched cationic polymer has a chemical structure:

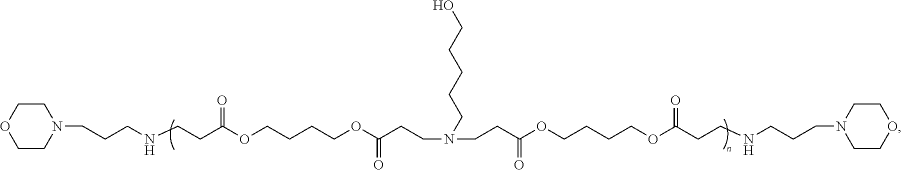

wherein n is between 1 and 500. These are referred to as E17 to E20 in the specification.

In one aspect, the hyperbranched cationic group is a β-amino ester.

In one aspect, the extraction microchannel (11) is connected to the third microchannel (9) in the second layer (3) at a junction (13) located between the elution buffer inlet port (5) and the eluate collection port (7).

In one aspect, the first layer (2) and the second layer are composed of a polymer.

Preferably, the polymer is selected from polycarbonate, polystyrene, polypropylene, poly(methyl-methacrylate), polydimethylsiloxane, polyurethane, polyethylene terephthalate, cellulose, nitrocellulose, polylactic acid, polycaprolactone, cyclic olefin copolymer, cyclic olefin polymer, or a combination thereof.

In one aspect, the nucleic acid is labelled and extracted at a purity of greater than 90%.

In one aspect, there is provided a method for extracting nucleic acid from a biological sample, the method comprising:
applying the biological sample to the sample inlet port (4) of the microfluidic chip (1) described herein,
applying a wash buffer and an elution buffer to the wash buffer inlet port (10) and the elution buffer inlet port (5), respectively,
applying pressure through the sample inlet port (4) of the microfluidic chip (1), and
collecting, by pressure driven flow, the extracted nucleic acid from the eluate collection port (7).

In one aspect, the method further comprises the step of incubating the biological sample on the hyperbranched cationic group-modified silica membrane in the membrane-containing chamber (6) of the microfluidic nucleic acid extraction chip (1) for a sufficient time prior to the step of applying the wash buffer and/or elution buffer.

In one aspect, the incubation period is for between about 5 minutes to about 120 minutes.

In one aspect, when the biological sample is incubated with the hyperbranched cationic group-modified silica membrane in the membrane-containing chamber (6) for selective DNA binding, a pressure of between about 0.01 bar to about 10 bar is applied to force the nucleic acid from the first layer (2) through to the second layer (3) of the chip (1) and eluted out.

In one aspect, the ratio of biological sample volume (SV) to wash buffer volume (BV) is 1:1 to about 1:8.

In one aspect, the biological sample volume and the wash buffer volume are equal.

In one aspect, the elution buffer is applied to the elution buffer inlet port (5) between 1 to 4 times per extraction. Preferably, the elution buffer is applied to the elution buffer inlet port (5) between 1 to 2 times per extraction.

In one aspect, the extracted nucleic acid has a purity of greater than 90%.

In one aspect, the nucleic acid is a polymeric molecule, incorporating units of ribonucleic acid (RNA) or an analog thereof, or deoxyribonucleic acid (DNA) or an analog thereof.

In one aspect, the biological sample is selected from blood, blood plasma, blood components, saliva, urine, cerebrospinal fluid, urine, neonatal fluid, aspirate, and the like.

In one aspect, the method is adapted for use with a nucleic acid amplification device. In one aspect, the nucleic acid amplification device is a polymerase chain reaction (PCR) device, a strand displacement assay (SDA) device, or a transcription-mediated assay (TMA) device.

In one aspect, there is provided a method of modifying the silica membrane in the membrane-containing chamber (6) of the microfluidic chip (1) described herein with a hyperbranched cationic group, the method comprising the steps of oxidising the silica membrane surface by applying an ultraviolet light to the surface; and curing the ultraviolet oxidised surface using a silanizing agent in an HPAE-solution to form the hyperbranched cationic group-modified surface.

In one aspect, the step of applying the ultraviolet light is for between 15 to 120 minutes.

In one aspect, the curing step further comprises incubating the silica membrane with acetic acid.

In one aspect, the acetic acid is at a concentration of about 20 to about 100 mM.

In one aspect, the HPAE solution is at a concentration of between 1 mg/ml and 50 mg/mL.

In one aspect, the cationic group is selected from an amino ester, sulphonium, oxonium, phosphonium, imidazolium, pyrrolidinium, pyridinium, pipyridinium, morpholinium, and quinolinium. Preferably, the catanionic group is a β-amino ester.

In one aspect, there is provided a kit for extracting nucleic acid from a biological sample, the kit comprising the microfluidic nucleic acid extraction chip described herein, an elution buffer, and a wash buffer.

In one aspect, the elution buffer comprises Tris-ethylene diamene tetra-acetic acid adjusted to pH>7.

In one aspect, the wash buffer comprises a non-ionic surfactant (for example, Triton X-100 (C₁₄H₂₂O(C₂H₄O)ₙ (ₙ=₉₋₁₀))), guanidine isothiocyanate and tris hydrochloride.

There still exist some severe problems in both laboratory and commercially oriented microfluidic DNA extraction platforms: i) Scaling up of complex silica structures such as microspheres, nanowires, micropillars inside the channels of the chip. The manufacturing and quality control costs increase with increasing complexity of structures. Some integration is not possible. ii) Improving the purity and yield of nucleic acids without conventional mechanical steps like centrifugation, which is difficult to integrate onto a microfluidic chip. iii) Achieving rapid extraction with minimal steps and without the use of solvents that can damage microstructures.

The MF chip and method of the claimed invention overcomes at least one of the above-mentioned problems. The modified membrane used in the MF chip of the claimed invention is embedded between two layers of plastic substates that contain bonded flow channels. This enables a rapid extraction (dual capture of nucleic acid from the conventional Si-membrane capture as well as the cationic polymer) with minimal steps, and the purified nucleic acid can either be collected or allowed to flow into an amplification chamber of a nucleic acid amplification system (such as a real time PCR instrument) for direct, near-patient pathogen monitoring. Further, the MF chip of the claimed invention can be integrated with an on-chip PCR/LAMP or other amplification systems for direct detection of a pathogen from a biological sample, such as blood. The on-chip membrane-based extraction method does not require a centrifugal/magnetic component as the nucleic acid capture and release is a biochemical interaction between a highly cationic synthesized membrane, and the nucleic acid from, for example, a lysed blood sample of ultra-small volume (about 50 μL). The microfluidic chip is a disposable one-use device, to minimize contamination and improve the efficiency of the overall process. This MF chip is made by plastics (polycarbonate (PC), polystyrene (PS), polypropylene (PP), poly(methyl-methacrylate) (PMMA), cyclic olefin copolymer (COC), cyclic olefin polymer (COP), etc.) through injection moulding, which are bio-compatible, recyclable, low cost and disposable.

### Definitions

In the specification, the term “nucleic acid” or “nucleic acid sequence” should be understood to mean any molecule, preferably a polymeric molecule, incorporating units of ribonucleic acid (RNA), deoxyribonucleic acid (DNA), or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the template nucleic acid is DNA. In another aspect, the template is RNA. Suitable nucleic acid molecules are DNA, including genomic DNA, ribosomal DNA and cDNA. Other suitable nucleic acid molecules are RNA, including mRNA, rRNA and tRNA. The nucleic acid molecule can be naturally occurring, as in genomic DNA, or it may be synthetic, i.e., prepared based upon human action, or may be a combination of the two. The nucleic acid molecule can also have certain modification such as 2′-deoxy, 2′-deoxy-2′-fluoro, 2′-O-methyl, 2′-O-methoxyethyl(2′-O-MOE), 2′-O-aminopropyl(2′-O-AP), 2′-O-dimethylaminoethyl(2′-O-DMAOE), 2′-O-dimethylaminopropyl(2′-O-DMAP), 2′-O-dimethylaminoethyloxyethyl(2′-O-DMAEOE), or 2′-O—N-methylacetamido (2′-O—NMA), cholesterol addition, and phosphorothioate backbone as described in U.S. patent application No. 20070213292; and certain ribonucleoside that are linked between the 2′-oxygen and the 4′-carbon atoms with a methylene unit as described in U.S. Pat. No. 6,268,490, wherein both patent and patent application are incorporated hereby reference in their entirety. Example nucleic acid agents include, but are not limited to, a nucleic acid encoding a protein inhibitor (e.g. transcriptional inhibitors), oligonucleotides, nucleic acid analogues (e.g. peptide-nucleic acid (PNA), pseudo-complementary PNA (pc-PNA), locked nucleic acid (LNA) etc.), antisense molecules, ribozymes, small inhibitory or activating nucleic acid sequences (e.g. RNAi, shRNAi, siRNA, micro RNAi (mRNAi), antisense oligonucleotides etc.).

In the specification, the term “hyperbranched” should be understood to mean organic macromolecules that are highly branched and have a dendritic architecture with a large degree of terminal groups.

In the specification, the term “microfluidic chip” and “microfluidic nucleic acid extraction chip” should be understood to be the same chip, and the two terms can be used interchangeably.

In the specification, the term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

### BRIEF DESCRIPTION OF THE DRAWINGS

The invention will be more clearly understood from the following description of an embodiment thereof, given by way of example only, with reference to the accompanying drawings, in which:—

FIG. 1 is an illustration of direct capture of nucleic acid from a clinical specimen using the claimed invention for rapid, low volume detection on point-of-care devices.

FIG. 2(A) is a series of graphs illustrating the characterization of synthesized polyamine esters (PAEs), while FIG. 2(B) are gel permeation chromatography (GPC) traces of ¹H NMR spectra of the synthesized PAEs.

FIG. 3 illustrates the effect of surface oxidation and silanization through UV and PTMS treatment on DNA yield and purity. (A) (3-Glycidyloxypropyl)trimethoxysilane (GPTMS) treatment of oxidized Si surface forming silanol bonds; (B) Primary amines (a) and secondary amines (b) react with epoxy groups of GPTMS; (C) Absorbance ratio of DNA to contaminants after extraction using membranes with or without 10% GPTMS retained over time; (D) Increasing GPTMS % in the membrane improves DNA capture as absorbance ratio improves during extraction and higher resultant yield; (E) Exposed oxides on Si groups on membrane surface; and (F) Increase in time for oxidation increases purified DNA yield and absorbance due to increased cationic groups on the surface.

FIG. 4 shows analysis of commercial (uncoated Si membrane) and HPAE-modified (modified Si membrane) confirming (A) the presence of Amine and ester groups in Si membrane modified by E12 to E20 HPAEs. (B) are SEMs of the polymer structures on the modified surface of membrane fibers observed and compared against smooth membrane fibers (commercial).

FIG. 5 a schematic design of the microfluidic chip of the claimed invention, showing (A) a first (top) side and (B) a second (bottom) side. The chip also comprises the membrane region and flow regions. Sample is pumped through the inlet port in the first (top) layer into the embedded PAE-silica-membrane and filtered into the second (bottom) layer.

FIG. 6 shows the testing of the bonded microfluidic chip of FIG. 5 with a PAE-modified membrane: (A) dye injection to test against leakage/blocks in microchannels; (B) DNA extraction assay: 50 μL sample and 100 μL wash buffer are injected into the modified membrane via ports and incubated for 15 minutes at 56° C. before elution; (C) increasing cationic binding sites in higher PAE molecular weight results in increased yield for branched polymers; (D) effect of PAE concentration in membrane modification solution on quality of extractant.

FIG. 7 shows the recovery and purity of extracted DNA in graph form for (A) lysis conditions compared to Qiagen, (B) varying ratio of spiked blood volume and buffer, (C) elution buffer volumes, (D) flow rate, (E) CMV Fluorescence of Loop Primer Upon Self Dequenching-Loop mediated isothermal Amplification (FLOS-LAMP) assay signal intensity curves of standard and PAE-modified membrane DNA extract, and (F) performance of extracted DNA using CMV FLOS-LAMP assay with lowest limit of detection at 300 IU/mL viral load.

### DETAILED DESCRIPTION OF THE DRAWINGS

Poly amine esters (PAEs) are widely used in gene therapy and drug delivery. Compared to the conventional synthetic polymers used in DNA capture that always require a strict or complex polymerization process, PAEs are easier to synthesize. Synthesis can be achieved by facile Michael addition, making it more suitable for large scale manufacture. Meanwhile, the abundance of inexpensive monomers and easily modified terminal groups allows for tuning polymer chemical structure and topology according to application requirements. In addition, the DNA purity and concentration are measured by calculating the absorption ratio at 260 nm (conjugated purines and pyrimidines groups) and 280 nm (conjugated amino acids). As the PAE backbone does not have a conjugated structure, it does not interfere with absorption measurements at shorter wavelengths, thus residual coating polymer in the DNA extract does not affect the absorption values. To construct PAEs with high DNA capture capability, the monomer combination of 1,4-butanediol diacrylate (B4), 5-amino-1-pentanol (S5), and 3-Morpholinopropylamine (MPA) was selected. According to previous research, the combination of hydrophobic B4 and hydrophilic S5 can facilitate strong DNA binding capacity while maintaining stability in the aqueous environment. Meanwhile, the morpholinopropylamine (MPA) is a group proven to have strong DNA interaction ability. The terminal modification of MPA can enhance the DNA binding capability of the PAEs. Moreover, a branch unit, pentaerythritol tetra-acrylate (PTTA), was introduced to achieve a hyper-branched PAE, resulting in higher number of terminal group. The transition from a linear to a branched structure enhances the DNA binding ability of PAEs, which is particularly important given the difficulty in achieving high molecular weight linear PAEs with a limited number of terminal groups.

The Applicants present a novel application of hyperbranched poly(ß-amino ester) (HPAEs) in modifying the surface of silica membrane to increase electrostatic capture of DNA (for example) from low volume blood plasma (for example) on a microfluidic device. This application adds to the aforementioned advantages of silica membrane and facilitates extraction without the need for organic solvents or additional instruments (see FIG. 1). HPAE is attached onto the silica fibres in membrane using an organosilane crosslinking agent, which can then be easily embedded in between two layers of polymethyl methacrylate (PMMA) chips for extraction. The resulting extraction chip can process the sample in an instrument-free, direct (two steps, no lysis or other pretreatment) and rapid (<20 minutes) manner to integrate with an amplification component for pathogen detection. By bonding multiple layers of PMMA with the HPAE-modified membrane embedded, the plasma sample can be incubated with the membrane for selective DNA binding on the top layer and filtered through to the bottom layer and eluted out by pressure driven flow. The assay optimization of this membrane-based device does not contain organic solvents such as ethanol, thus removing the possibility of amplification inhibitors. The highly cationic nature of the modified membrane is significant for the direct binding and elution of DNA without the use of multiple components. The eluted DNA is amplified by using real time loop mediated isothermal amplification and the fluorescent signals are recorded.

### Materials and Methods

### Polymer Synthesis for Membrane Coating:

### PAE Materials Synthesis

Chemicals: 1,4-butanediol diacrylate (B4), 5-amino-1-pentanol (S5), 3-Morpholinopropylamine (MPA), Pentaerythritol tetra-acrylate (PTTA), lithium bromide (LiBr), deuterated chloroform (CDCl3), and dimethyl sulfoxide (DMSO) were purchased from Sigma Aldrich (UK). Solvent dimethylformamide (DMF), and diethyl ether were purchased from Fisher. Poly(ß-amino ester) s (PAEs) was synthesized using different amounts of monomers (see Table 1) dissolved in DMSO and the reaction was performed at 90° C. Gel permeation chromatography (GPC) was used to monitor the growth of molecular weight. Once the weight molecular weight (Mw) reached target value, the reaction was further diluted with DMSO and end-capped with MPA. Finally, the polymer product was precipitated in diethyl ether, and dried under vacuum to remove solvent residue.

TABLE 1
Monomer amount for PAEs syntheses

| | B4 (g) | PTTA (g) | S5 (g) | MPA (g) |
|---|---|---|---|---|
| E12 | 0.99 | 0.18 | 0.48 | 1.12 |
| E13 | 0.99 | 0.18 | 0.49 | 1.08 |
| E14 | 0.99 | 0.18 | 0.50 | 0.95 |
| E15 | 0.99 | 0.18 | 0.52 | 0.86 |
| E16 | 0.99 | 0.09 | 0.52 | 0.86 |
| E17 | 1.19 | 0 | 0.52 | 0.86 |
| E18 | 1.19 | 0 | 0.56 | 0.84 |
| E19 | 1.19 | 0 | 0.60 | 0.84 |
| E20 | 1.19 | 0 | 0.62 | 0.84 |

### Molecular Weight Measurements

Number average molecular weight (Mn), weight average molecular weight (Mw), and the polydispersity index (PDI) of polymers were determined by GPC equipped with a refractive index detector (RI), a viscometer detector (VS DP) and a dual angle light scattering detector (LS 15° and LS) 90°. To monitor the Mw of polymers during the polymerization process, 20 μL of the reaction mixture was collected at different time points, diluted with 1 mL of DMF, filtered through a 0.2 μm filter and then measured by GPC. The two in-series columns were eluted with DMF and 0.1% LiBr at a flow rate of 1 mL/min at 60° C.

### Proton Nuclear Magnetic Resonance (¹H NMR)

Chemical structure and composition of polymers were confirmed with ¹HNMR. PAEs were dissolved in CDCl₃. Measurements were carried out on a Varian Inova 400 MHz spectrometer and reported in parts per million (ppm).

### Membrane Preparation:

Commercial silica membrane sheets for nucleic acid extraction with pore size of 1 μm and thickness 1 mm were purchased from Biocomma Ltd. (Shenzhen, China). The membranes were cut, washed, and dried with 70% ethanol (Fisher Scientific). To modify the surface, (3-Glycidyloxypropyl)trimethoxysilane (GPTMS) and Acetic Acid were purchased from Sigma Aldrich (UK). All experiments were carried out in a Biobase BSL-II laminar air flow chamber. The surface of the commercial Si membrane was oxidised by UV exposure, followed by direct adsorption of the polymer solution containing HPAE polymer and the crosslinker in varying amounts (Wang, X., et al., Integrated silica membrane-based nucleic acid purification, amplification, and visualization platform for low-cost, rapid detection of foodborne pathogens. Analytical and Bioanalytical Chemistry, 2020. 412 (25): p. 6927-6938; and Shirosaki, Y., et al., In vitro cytocompatibility of MG63 cells on chitosan-organosiloxane hybrid membranes. Biomaterials, 2005. 26 (5): p. 485-493). After curing the membrane under UV, the excess PAE solution was washed out and dried overnight in a desiccant chamber. To identify optimum conditions under which the surface modification of the polymers on the membrane was durable and able to extract DNA, surface oxidation time, GPTMS amount in polymer solution and polymer concentration were all varied (see Table 1). Characterisation of the modified membrane was performed using Bruker ATR FTIR system and imaged on Zeiss Sigma 300 SEM.

TABLE 1
Coating conditions for PAE-modified Si membrane for extraction

| Parameters | Values |
|---|---|
| UV exposure | 15 min/30 min/1 hour/2 hours |
| GPTMS concentration | 0%/1%/5%/10% |
| Polymer concentration | 1/2/10/20/40 mg/mL |

### Microfluidic Chip Fabrication & Assembly:

The chips were laser cut from industrial grade PMMA sheets (Radionics Ltd.) and milled to a tolerance of ±100 μm in the microchannels using a Microcut 873 milling machine. Analytical grade Isopropanol and Chloroform were purchased from Sigma Aldrich for the ultrasonication bath and thermal bonding respectively. Polymer-polymer bonding of the cleaned chips was performed in a precision hot press (MiNAN Technologies). Milled PMMA chips were first cleaned in an isopropanol bath under ultrasonication for 3 minutes and dried using an air gun. The bonding sides were exposed to UV/ozone treatment for 5 minutes followed by direct chloroform deposition for 2 minutes. Immediately afterwards, the bonded layers were subjected to high pressure bonding at 6000N at 63° C. for 10 mins.

### Extraction Assay and Fluorescent LAMP Based Detection:

Washing and elution buffer ingredients (Guanadium Isothiocyanate, Tris-HCl, Triton-X, EDTA) of molecular grade were purchased from Sigma Aldrich (UK). Blood plasma was obtained from the National Virus Reference Laboratory, Dublin, Ireland and the plasma was spiked to the required viral load using Acrometrix CMV Standards (300 to 300,000 IU/mL). Total genomic DNA was extracted using Qiagen (ThermoFisher) QiaAMP Virus MiniElute Spin Kit. Absorption at 260 nm and 280 nm for assessing DNA purity and yield were measured using Thermo Scientific® NanoDrop® Lite Spectrophotometer. DNA purity % and DNA recovery % were estimated five times for each condition and recorded with the standard deviation according to the equations below.

$$\% DNA Purity = \frac{( \frac{A 260}{280} ) HPAE - Si membrane}{( \frac{A 260}{280} ) Spin column membrane} \times 100 \qquad ( 1 )$$

$$\% DNA Recovery = \frac{DNA in ng per μ L of eluted volume}{DNA in ng per μ L of plasma} \times dilution factor \times 100 \qquad ( 2 )$$

For LAMP-based detection, Warmstart fluorescent LAMP mastermix and primers were purchased from New England Biolabs, UK and the primers were designed using the NEB LAMP Primer Design Toolkit to target the viral DNA polymerase region UL54 which is inhibited by the activated first-line therapeutic drug, ganciclovir, thus frequently used in CMV infection treatment. The loop forward primer was fluorescein tagged at the 3′ end for signal detection. Nuclease Free Water (Molecular Assay grade) purchased from ThermoFisher was used for all assays. Applied Biosystems® QuantStudio® 7 Flex Real-Time PCR System was used to detect the FAM-6 signal. The amplification program was run for 45 minutes at 65° C. followed by heating at 85° C. for 5 minutes. Two sets of standard positive controls (Acrometrix CMV 3000 IU/mL) extracted using Qiagen MiniElute and no template control (nuclease free water) were run during each assay and the amplification signals were recorded on QuantStudio QS7 Real Time PCR Software. All buffers were adjusted for their appropriate pH, using a digital pH meter. Assay optimization for on-chip process was performed to identify factors of significance, while the membrane size was fixed (see Table 3).

TABLE 2
On-chip CMV DNA extraction from spiked blood
plasma (A) Chemical composition of washing and elution
buffers; (B) Assay optimization factors

| A | Washing buffer | Elution buffer |
|---|---|---|
| | GuSCN | 4M |
| | Triton-X | 20 g/L |
| | Tris HCl | 10 mM |
| | pH adjusted to 6.5 | pH adjusted to 8.5 |
| | (1N HCl) | (1M NaOH) |
| B | Parameters | Values |
| | Viral load | 3000 IU/mL in blood plasma |
| | Input blood volume | 20/50/100/200 μL |
| | Sample-to-buffer ratio | 1:1/1:2/1:4 |
| | Incubation | 20 °C/56 °C |
| | Elution buffer volume | 50/100/200 μL |
| | Flow rate | 20/40/80/160 L/min |

### Results and Discussion

### Polymer Synthesis & Characteristics:

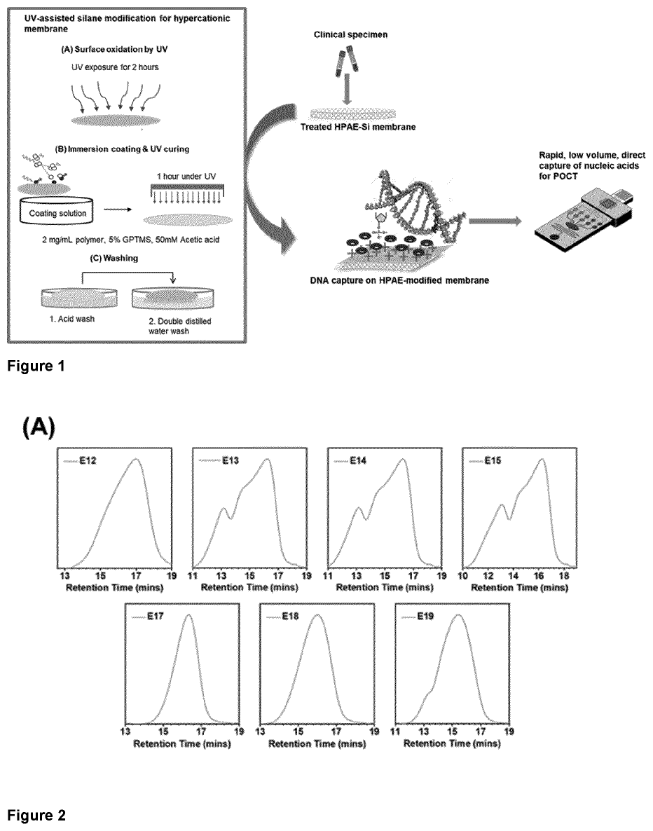

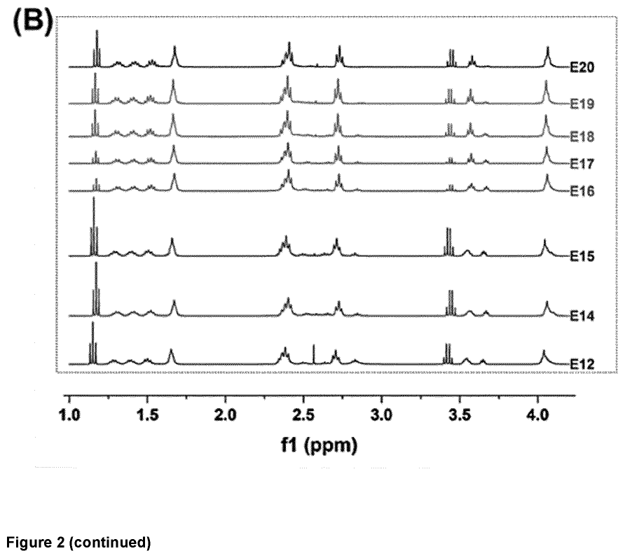

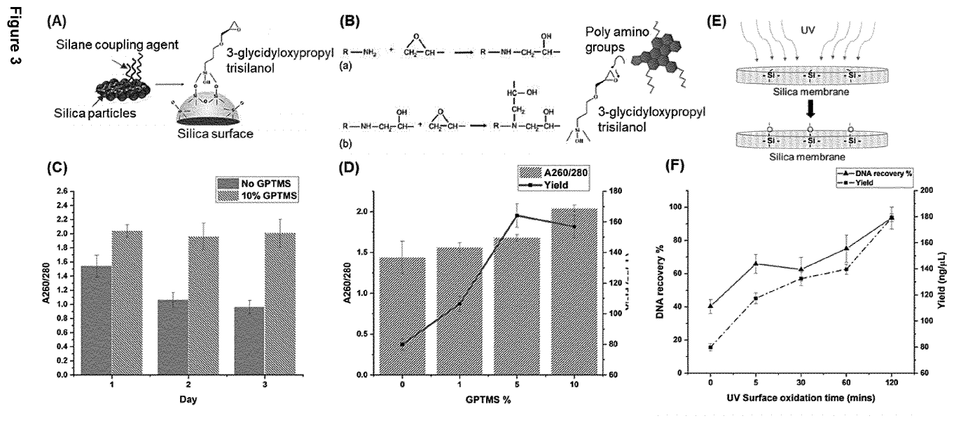

To tailor the properties of the poly(ß-amino ester) s (PAEs) for the claimed invention, a series of HPAEs, PAE-E12 to E19 with the branch ratio (branch monomer to the linear monomer molar ratio) as 0.1 were designed (see Scheme 1 below). According to the GPC results (see FIG. 2 and Table 4), their molecular weights (Mw) range from 10.7 kDa to 64.0 kDa. Meanwhile, a groups of linear PAE, PAE-E17 to E20 with molecular weights (Mw) ranging from 5.3 kDa to 21.1 kDa were synthesized for comparison. The polymer structures and monomer combinations were confirmed by NMR (see FIG. 2B and Table 4A). The NMR analysis shows that the amount of terminal amines (MPA) increases in proportion to the Mw in the branched PAEs (E12 to E15), while the opposite trend is observed in the linear PAEs (E17 to E20). Since the terminal amines have abundant nitrogen and oxygen groups, they have a higher chance of interacting with the DNA and silica membrane.

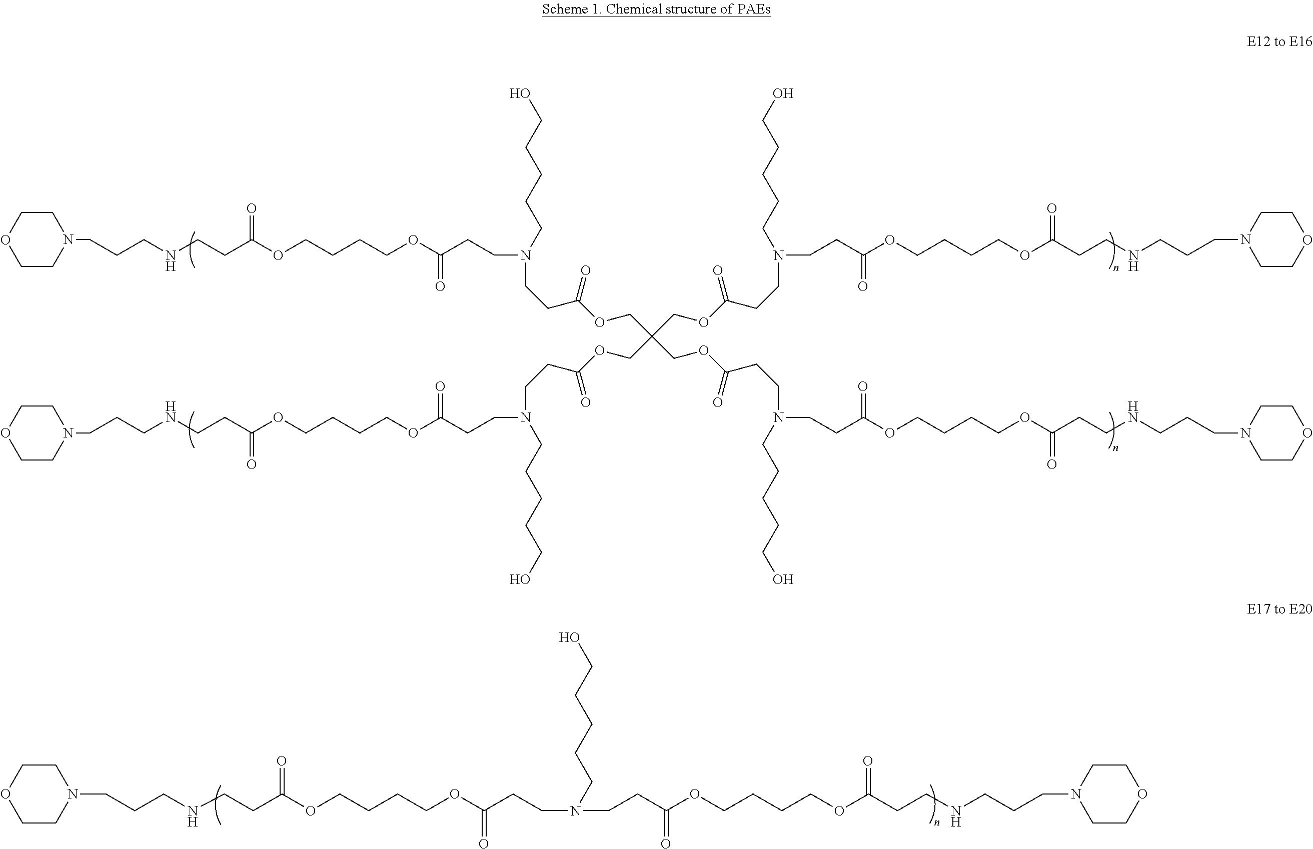

TABLE 3
(A) Structure properties of PAEs and
(B) Monomer molar ratios of HPAEs

| A | | Mn | Mw (Da) | PDI | Branch ratio |
|---|---|---|---|---|---|
| | E12 | 4475 | 10707 | 2.4 | 0.1 |
| | E13 | 6447 | 38170 | 5.9 | 0.1 |
| | E14 | 6289 | 42246 | 6.7 | 0.1 |
| | E15 | 6641 | 63991 | 9.6 | 0.1 |
| | E16 | 3112 | 4763 | 1.5 | 0.05 |
| | E17 | 3555 | 5294 | 1.5 | 0 |
| | E18 | 4513 | 7996 | 1.8 | 0 |
| | E19 | 6938 | 21125 | 3.0 | 0 |
| | E20 | 6524 | 21910 | 3.3 | 0 |
| B | | B4 (g) | PTTA (g) | S5 (g) | MPA (g) |
| | E12 | 1 | 0.1 | 0.94 | 0.28 |
| | E13 | 1 | 0.1 | 0.96 | 0.32 |
| | E14 | 1 | 0.1 | 0.98 | 0.38 |
| | E15 | 1 | 0.1 | 1 | 0.40 |
| | E16 | 1 | 0.05 | 1 | 0.20 |
| | E17 | 1 | 0 | 0.83 | 0.34 |
| | E18 | 1 | 0 | 0.91 | 0.18 |
| | E19 | 1 | 0 | 0.97 | 0.06 |
| | E20 | 1 | 0 | 1 | 0.03 |

### HPAE-Modified Si Membrane Characteristics:

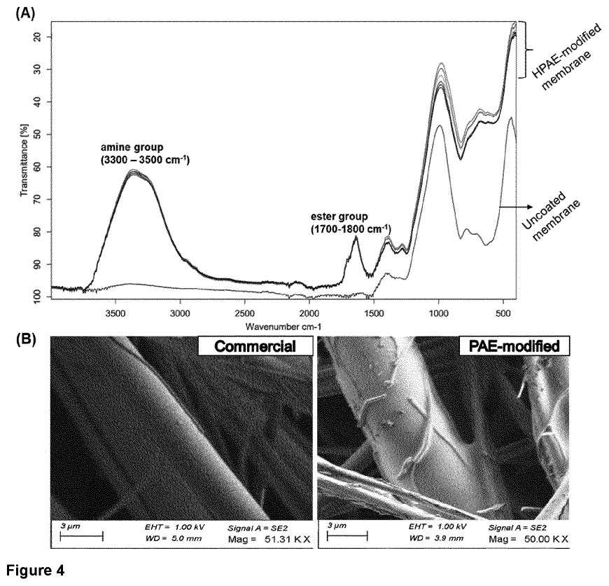

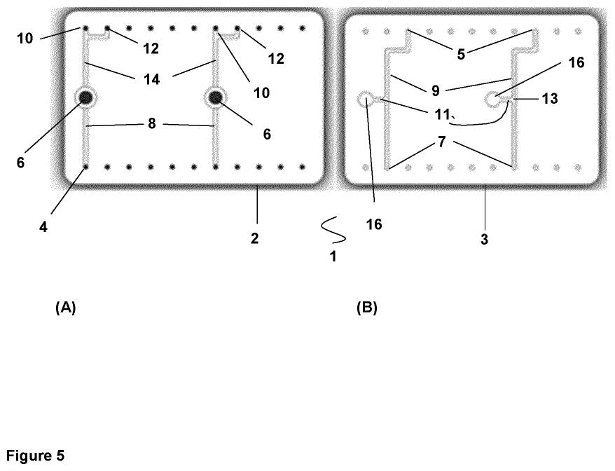

The epoxy groups on the GPTMS form a covalent bond with the primary and secondary amines of the HPAE polymers (see FIGS. 3A and 3B) and bind to an oxidized silica surface. It was found that the volume of the crosslinker GPTMS in the HPAE-polymer solution is significant for the shelf life of the modified membrane. Without any GPTMS, the recovery and purity of DNA reduces by 50%, while DNA recovery increases with an increase in % GPTMS (see FIG. 3(C)). The increase in GPTMS volume leads to a higher number of cationic groups on the Si membrane, providing more binding sites for the negatively charged phosphate backbone of DNA (see FIG. 4(D)). However, using >10% GPTMS was found to inhibit downstream amplification processes, resulting in the signal intensity of CMV LAMP being below threshold for DNA detection. Despite relatively high yield of DNA (see FIG. 3(D)), a cloudy precipitate was observed in the extracted DNA at 10% GPTMS, which could be the excess silanes. To avoid this issue and not affect LAMP detection, further experiments were carried out using 5% GPTMS.

Oxidation of silica membrane can be carried out by chemical methods such as treating the surface with piranha solution or physical methods like UV exposure. Exposure of silica fibers to UV for prolonged time increases the number of oxidized Si groups on the surface (FIG. 3(E)). This, in turn, promotes polymer binding on adsorption with the crosslinker. The linear increase in DNA absorbance and yield is observed as surface oxidation time of membranes before HPAE coating. With higher exposure time to UV, the number of oxidized silica sites on the membrane increased, enabling a greater number of GPTMS-linked HPAE molecules to attach onto the surface through their epoxy groups. This is further supported by the increase in DNA yield from 117.43 ng/μL to 179.18 ng/μL, as shown in FIG. 3(F).

ATR-FTIR spectra confirmed the presence of amine and ester groups on membranes modified using all the polymers (E12 to E20) using 5% GPTMS, 2 hours UV exposure and 5 mg/ml polymer concentration. The FTIR analysis was repeated for 5 days consecutively to observe any reduction in transmittance peaks at 3300-3500 cm-1 and 1700-1800 cm-1 and was observed to be consistent. As the number of amine groups was significantly higher than the esters, the transmittance peak at wavelengths corresponding to primary and secondary amine groups were also much higher (FIG. 4(A)). SEM analysis of membrane fibers before and after modification revealed fold-like structures on the surface of the fibers which were smooth before modification. These structures are likely the polymer particles attached on the surface after modification (FIG. 4(B)). To ensure there was no dust or contamination on the surface the membranes were modified in a sterilized laminar flow chamber and placed carefully between two clean glass slides before imaging. The imaging was repeated three times in two different SEM instruments to avoid any artefacts. Imaging of the same samples was done after one month to observe if the polymer structures remained on the surface; they were found to remain consistent.

### Microfluidic Assembly and On-Chip HPAE-Modified Membrane Extraction:

Referring now to FIG. 5, which illustrates a general embodiment of a microfluidic (MF) nucleic acid extraction chip of the present invention. Specifically, FIG. 5 illustrates a plan view of the MF nucleic acid extraction chip of the claimed invention and is generally referred to by reference numeral 1. The MF nucleic acid extraction chip 1 of the illustrated embodiment comprises a first (top) layer 2 (see FIG. 5(A)) and a second (bottom) layer 3 (see FIG. 5(B)). The first layer 2 comprises a sample inlet port 4 in fluid communication with a membrane-containing chamber 6 via a first microchannel 8. The first layer 2 further comprises a wash buffer inlet port 10 and an elution buffer waste collection port 12 both of which are in fluid communication with the membrane-containing chamber 6 via a second microchannel 14. The second layer 3 comprises an elution buffer inlet port 5 in fluid communication with an eluate collection port 7 via a third microchannel 9. The membrane-containing chamber 6 in the first layer 2 is in fluid communication with the second layer 3 through an extraction microchannel 11, that joins the third microchannel 9 at a junction 13 between the elution buffer inlet port 5 and the eluate collection port 7. The MF nucleic acid extraction chip 1 further comprises a reservoir 16 for storing the nucleic acid extracted from the membrane.

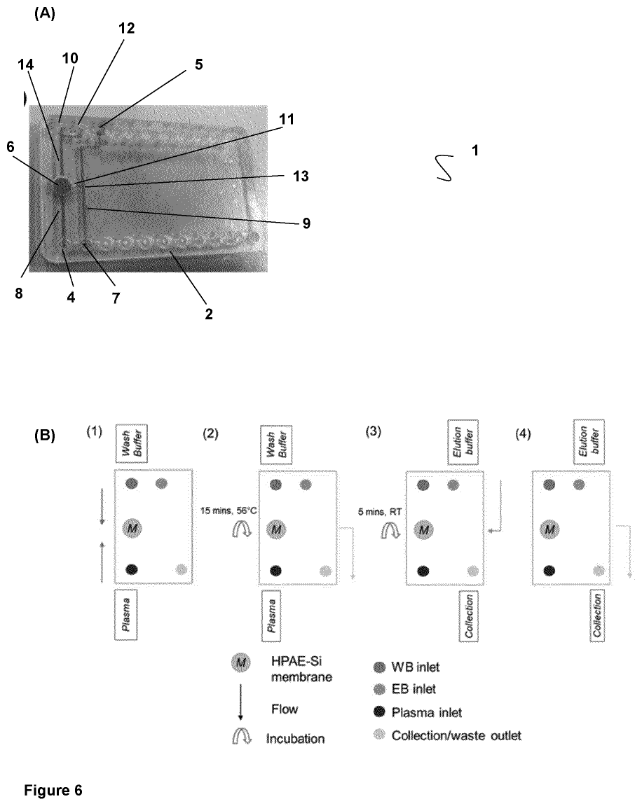

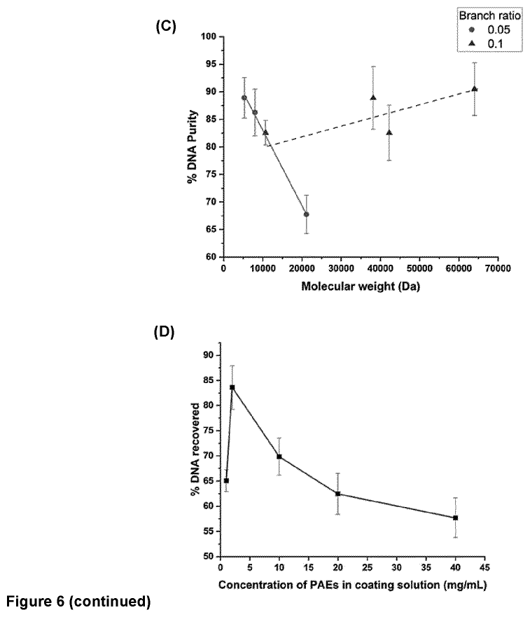

The bonded nucleic acid extraction MF chip 1 containing the membrane-containing chamber 6 (FIG. 5(A) and (B), and FIG. 6(A) was analyzed for fabrication defects using a standard microscope. To ensure there was no leakage or structural disturbances to the fluid movement within the sealed MF nucleic acid extraction chips 1, a locally purchased food dye was injected into the ports 4, 5, 7, 10 and the flow was tracked using a microscope (FIG. 6A). All the channels 8, 9, 11, 14 were properly filled, and the test was repeated over 4 separate bonded MF chips 1 for confirmation. Bonding strength was assessed using burst pressure test equipment. A manual water pressure test pump was used to pump water into the inlets ports 4, 10 of the MF chip 1 while blocking the outlet ports 5, 7, 12 with a plug. This plug was manually pressed during the test and a pressure gauge was used to show the burst pressure of the MF chip 1. Four different MF chip sets were assessed, and the bonded MF chips 1 did not produce leakage at a maximum pressure of 50 MPa.

On-chip extraction was carried out using blood samples spiked with Acrometrix CMV standards (3000 IU/mL). 50 μL of CMV spiked plasma samples were injected through the sample inlet port 4 using a sterile syringe and connecting tube. After incubation on a thermal heating block, 100 μL of elution buffer was pumped into the elution buffer inlet port 5 and the purified DNA was collected and analyzed using a Nanodrop Spectrophotometer (FIG. 6B).

To achieve good DNA extraction, the coating PAEs with different structures were screened. The evaluation of DNA purity on PAE-coated membranes revealed an essential relationship between coating-PAE's molecular weight and molecular structure with DNA extraction efficiency. FIG. 6(C) illustrates that in linear PAE-E17 to E19, the DNA purity decreased rapidly with the increase of molecular weight (as the molecular weight increased from 5k to 21k, the DNA extraction efficiency decreased from 88% to 67%), which was due to the reduction of the terminal amine group in high molecular weight PAEs. This molecular weight-dependent tendency indicated that the terminal functional groups of the polymer could significantly help DNA extraction. Specifically, the pending positive charged terminal amines can interact and bind DNA via electrostatic interaction. Since HPAE has more amino terminations than LPAE, different HPAE-coated membranes were investigated further. As expected, HPAE can significantly improve the DNA extraction efficiency of membranes-all the tested groups maintained more than 80% DNA purity. In addition, with the coated-HPAE molecular weight increased, the membranes' DNA extraction performance improved slightly (82% to 90%, Mw from 10k to 64k). However, the limited improvement in efficiency from E12 to E15 shows that the positively charged termination density in coating polymer is the main factor affecting DNA extraction performance. In comparison with LPAE, the positive charge density in HPAE does not change with the increase of molecular weight. Specifically, HPAE is not sensitive to molecular weight allowing for consistent performance across a wide range eliminating the need for precision instruments such as GPC to monitor large-scale production. In addition, HPAE has a shorter synthesis time compared with linear PAE. It is optimized to complete the HPAE polymerization reaction in hours, much faster than the days required for LPAE. Most importantly, the synthesis conditions of HPAE are mild (do not require high temperature/low temperature, high pressure/vacuum, or even protection of inert conditions). Consequently, no large specialized equipment is required to produce the coatings, making them easy to manufacture. Moreover, the reactant can have a reaction extent close to 100%, thus guaranteeing a very high yield. All these merits show that the HPAE-coated membrane can work as a promising DNA extraction device.

To optimize the coating conditions, E15 solutions with different concentrations were tested (FIG. 6(D)), and it was found that the PAE-based membrane exhibited the best performance after treatment with a 2 mg/mL solution. However, higher concentrations of PAE led to a decrease in DNA recovery, likely due to a higher number of attached polymer molecules, resulting in less DNA release during assessment.

### On-Chip Assay Optimization and FLOS-LAMP Detection:

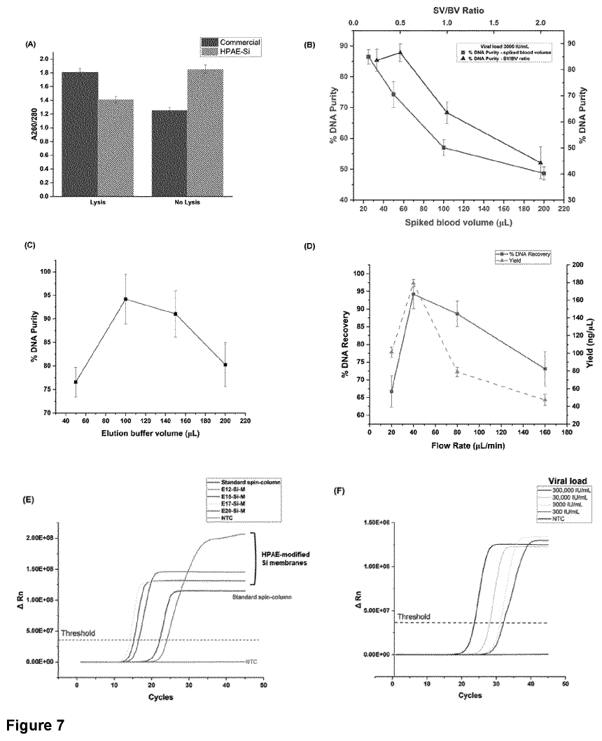

Commercial lysis reagents contain a detergent such as SDS or Triton-X that can break open cells for isolating DNA for downstream processes in molecular biology. Sodium dodecyl sulphate (SDS) is inhibitory in most amplification processes, and thus is not preferred for DNA extraction. Alternatively, Triton-X is a non-ionic detergent that can break cell membranes without affecting amplification processes. PAE-modified membrane-based DNA extraction was performed by directly treating 100 μL blood with an equal volume of washing buffer. The extraction method without pre-treatment with lysis buffer had a higher absorbance ratio (1.8) when compared to pre-treatment with a commercial lysis buffer (1.4) (see FIG. 7A). Lysis and DNA binding were combined as a single wash step using a chaotropic salt solution with Triton-X.

Blood sample volume and viral load can affect the amount of DNA available for binding and extraction due to the ratio of total DNA and contaminants (plasma components such as proteins). It was observed that for a fixed viral load of 3000 IU/mL, the purity of DNA recovered reduced with increases in sample volume (FIG. 7B). This could be due to the increased non-specific binding of circulating DNA in plasma on the cationic surface, while significant amounts of viral DNA are pumped out as waste. This was confirmed by collecting the filtrate waste and extracting DNA from it using Qiagen Spin Kit, and the DNA yield was 132 ng/μL. A critical metric in the assay development of the HPAE-Si membrane extraction was the ratio of sample volume to wash buffer volume (SV/BV). As the area of the membrane is fixed and therefore the number of open cationic binding sites is limited, the ideal pH and salt concentration conditions driving DNA towards the binding sites are influenced by the volume of the buffer relative to the blood volume. This was apparent as the SV/BV ratio increased, the buffer amount was insufficient to increase the positive charge density on the membrane surface and drive the exposed phosphate backbone of the DNA to bind. Thus, polymer cationic sites were occupied non-specifically by contaminant biomolecules, resulting in a much lower DNA purity for the same HPAE-modified membrane while an assay using SV/BV ratio of 0.5 on the HPAE-modified membrane resulted in >90% DNA purity (FIG. 7B). The volume of elution buffer used for one cycle was found to be optimal at 100 μL and contaminants increased with increasing elution buffer volume (FIG. 7C). However, in the case of viral loads<3000 IU/mL, increasing the number of elution cycles to 2 or 3 was found to increase DNA purity (from 77% to 92%) while the yield increased to 179 ng/μL.

After recording the absorbance ratios at 260 nm and 280 nm, and DNA yield, the extractant was used as template DNA for real time FLOS-LAMP detection of CMV. 5 μL of template DNA was added to the LAMP mix (12.5 μL Bst Warmstart mastermix, 2.5 μL primer mix, 5 μL nuclease free water). Comparing against the positive control extracted using Qiagen MiniElute Spin kit, the CMV positive signal from samples extracted on-chip using modified membranes (E12, E15, E17, E20) were above the fluorescence threshold and were detected (FIG. 7E). Each sample was amplified in triplicates and the mean cycle time to detection was found to be 12 minutes (E15, E17, E20 modified) and 20 minutes (E12 modified). This cycle time variation could be attributed to the increased specificity of DNA binding, as E15, E17 and E20 modified membranes were able to improve the A260/A280 ratio to 1.7±0.02 when compared to E12 (1.52±0.04) modified membrane. Performance evaluation of LAMP assay using the extracted sample as a template showed a lower limit of detection of 300 IU/ml of viral load (FIG. 7F). The assay was repeated in triplicates and the mean Ct value was recorded.

The on-chip modified silica membrane-based extraction and LAMP detection of CMV from blood was compared against commercial systems with integrated DNA extraction and amplification (Table 5). The purpose of this was to further the evidence of process simplification including time, instruments, sample pre-processing and manual steps.

TABLE 4
Semi-automated commercial nucleic acid extraction systems

| | Sample Type | Nucleic acid | | Detection & |
|---|---|---|---|---|
| Name | & Volume | purification | Manual steps | Turnaround Time |
| Cepheid GeneXpert | Sputum | Filter based | Vortexing during | Real time Reverse |
| MTB-RIF | (After lysis 2 | capture of | lysis. Transfer of | Transcriptase PCR with |
| | mL) | bacilli and | sample into Xpert | fluorescent detection. |
| | | ultrasonic | MTB/RIF cartridge | 2 hours |
| | | lysis | and loading the | |
| | | | cartridge into the | |
| | | | instrument | |
| Abbott ID Now | Throat Swab | No further | >1 cartridge | Real time isothermal |
| SARS-CoV2 | (200 μL | purification | transfers | amplification with |
| | Universal | | | fluorescent detection |
| | Transport | | | 13 mins |
| | Media) | | | |
| Biofire (BioMerieux) | Whole Blood | Binding on | No manual steps. | PCR detection with |
| Filmarray Blood | (200 μL) | surface | Each film array is | fluorescent probes. 1 |
| Culture ID Panel | | functionalized | for 1 sample, | Hour |
| | | magnetic | disposable and | |
| | | beads | does all 3 | |
| | | | (extraction, PCR, | |
| | | | detection) in the | |
| | | | instrument | |
| NucliSENS | Plasma | Adsorption on | Transfer of sample | NASBA amplification |
| EasyQ ® HIV-1 | (0.1 mL) | magnetic | from bead-based | with fluorescent probes |
| (BioMerieux) | | silica beads | extraction unit to | 3 hours |
| Automated Unit | | | amplification unit | |
| Including: | | | | |
| NucliSENS | | | | |
| easyMAG | | | | |
| configuration | | | | |
| NucliSENS EasyQ | | | | |
| configuration | | | | |
| Mini Strip | | | | |
| Centrifuge | | | | |
| Alethia CMV | Saliva Swab | No further | Vortexing | LAMP with fluorescent |
| (Meridian | (1 mL | purification | Transfer of | detection |
| Biosciences) | Universal | | processed sample | Under 1 hour |
| | Transport | | to instrument | |
| | Media) | | | |
| AmpliVue C. difficile | Stool | No further | Transfer of lysed | LAMP with fluorescent |
| (Quidel) | (50 μL with | purification | sample into | detection |
| | lysis buffer) | | reaction tube | Around 1.5 hours |
| | | | Placement of | |
| | | | reaction tube into | |
| | | | amplicon cartridge | |
| | | | Placement into | |
| | | | detection cassette | |
| PAE Modified | Plasma | Attachment on | Injection of sample | LAMP with fluorescent |
| membrane | (50 μL) | membrane | and buffers into port | detection (integrated on |
| | | and elution | Transfer of | chip/external) |
| | | | cartridge to heating | Extraction time-17-20 |
| | | | system | minutes |
| | | | | Total time to detection |
| | | | | (including LAMP)-1.5 |
| | | | | hours |

### CONCLUSION

A 2-step nucleic acid extraction MF chip and method was developed using HPAE-modified silica membranes and demonstrated for CMV detection from plasma. The synthesis and screening of HPAE coatings, modification of membrane and MF chip fabrication methods are described, and the performance of the method was compared against commercially available silica spin column kits. GPTMS % and UV curing time were varied to identify the ideal conditions for all the HPAEs used to coat the membranes to improve the coating process efficiency. Using HPAE-modified silica membranes, absorbance ratio (A260/280) of 1.89 and DNA recovery of 94% with an average yield of 179 ng/μL have been achieved from plasma without any pretreatment steps, thus providing a rapid and scalable nucleic acid extraction method that can be integrated with an on-chip amplification component. The fluorescent signal intensity detected from the LAMP assay using the extracted template, with a minimal viral load of 300 IU/mL, was comparable with conventionally extracted CMV DNA, with fewer manual steps and the need for no bulky instruments in a rapid manner (20 minutes). The application of HPAEs for nucleic acid capturing was demonstrated and provided an efficient method for low resource on-chip extraction for commercial sample-to-result diagnostic devices.

In the specification the terms “comprise, comprises, comprised and comprising” or any variation thereof and the terms “include, includes, included and including” or any variation thereof are considered to be totally interchangeable and they should all be afforded the widest possible interpretation and vice versa.

The invention is not limited to the embodiments hereinbefore described but may be varied in both construction and detail.

All publications, patents, patent applications and other references mentioned herein are hereby incorporated by reference in their entireties for all purposes as if each individual publication, patent or patent application were specifically and individually indicated to be incorporated by reference and the content thereof recited in full.

1. A microfluidic nucleic acid extraction chip (1), the chip (1) comprising a membrane-containing chamber (6) bonded between a first layer (2) and a second layer (3), wherein the first layer (2) comprises a sample inlet port (4) in fluid communication with the membrane-containing chamber (6) by a first microchannel (8), and a wash buffer inlet port (10) and an elution buffer waste collection port (12) in fluid communication with the membrane-containing chamber (6) by a second microchannel (14); wherein the second layer (3) comprises an elution buffer inlet port (5) in fluid communication with an eluate collection port (7) adapted to deliver extracted nucleic acid to a nucleic acid amplification module via a third microchannel (9); and wherein the membrane-containing chamber (6) is in fluid communication with the second layer (3) through an extraction microchannel (11); and wherein the membrane-containing chamber (6) contains a silica membrane modified with a hyperbranched cationic polymer.

2. The microfluidic nucleic acid extraction chip (1) according to claim 1, wherein the hyperbranched cationic polymer has a cationic group selected from an amino ester, sulphonium, oxonium, phosphonium, imidazolium, pyrrolidinium, pyridinium, pipyridinium, morpholinium, and quinolinium.

3. The microfluidic nucleic acid extraction chip (1) according to claim 1, wherein the hyperbranched cationic polymer has a branch monomer to the linear monomer molar ratio of 0 to 0.1.

4. The microfluidic nucleic acid extraction chip (1) according to any claim 1, wherein the hyperbranched cationic polymer has a molecular weight range of between about 5 kDa to about 65 kDa.

5. (canceled)

6. The microfluidic nucleic acid extraction chip (1) according to claim 1, wherein the hyperbranched cationic polymer is composed of a mixture of two or more of 1,4-butanediol diacrylate, 5-amino-1-pentanol, 3-morpholinopropylamine, and pentaerythritol tetra-acrylate.

7. The microfluidic nucleic acid extraction chip (1) according to claim 1, wherein the hyperbranched cationic polymer has a 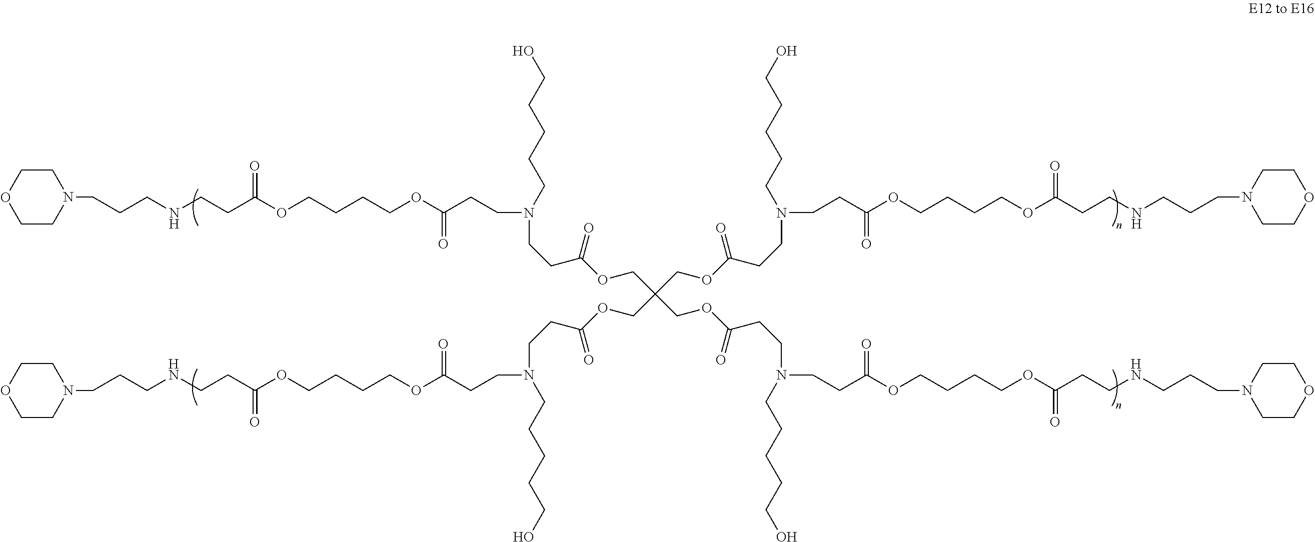
chemical structure:
wherein n is the number of the repeating monomer unit between 1 and 500.

8. The microfluidic nucleic acid extraction chip (1) according to claim 1, wherein the hyperbranched cationic polymer has a chemical structure: 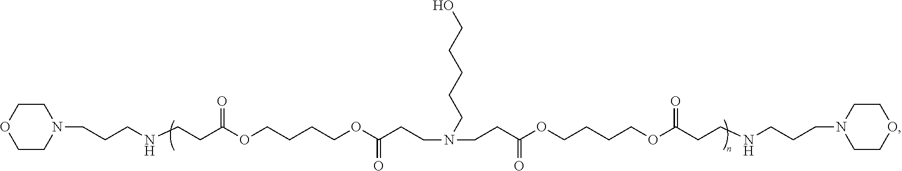
wherein n is between 1 and 500.

9-12. (canceled)

13. The microfluidic nucleic acid extraction chip (1) according to claim 1, wherein the nucleic acid is labelled and extracted at a purity of greater than 90%.

14. A method for extracting nucleic acid from a biological sample, the method comprising:
applying the biological sample to the sample inlet port (4) of a microfluidic chip (1) comprising a membrane-containing chamber (6) bonded between a first layer (2) and a second layer (3), wherein the first layer (2) comprises a sample inlet port (4) in fluid communication with the membrane-containing chamber (6) by a first microchannel (8), and a wash buffer inlet port (10) and an elution buffer waste collection port (12) in fluid communication with the membrane-containing chamber (6) by a second microchannel (14); wherein the second layer (3) comprises an elution buffer inlet port (5) in fluid communication with an eluate collection port (7) adapted to deliver extracted nucleic acid to a nucleic acid amplification module via a third microchannel (9); and wherein the membrane-containing chamber (6) is in fluid communication with the second layer (3) through an extraction microchannel (11); and wherein the membrane-containing chamber (6) contains a silica membrane modified with a hyperbranched cationic polymer,
applying a wash buffer and an elution buffer to the wash buffer inlet port (10) and the elution buffer inlet port (5), respectively,
applying pressure through the sample inlet port (4) of the microfluidic chip (1), and
collecting, by pressure driven flow, the extracted nucleic acid from the eluate collection port (7).

15. The method according to claim 14, further comprising the step of incubating the biological sample on the hyperbranched cationic polymer-modified silica membrane in the membrane-containing chamber (6) of the microfluidic nucleic acid extraction chip (1) for a sufficient time prior to the step of applying the wash buffer and/or elution buffer.

16. (canceled)

17. The method according to claim 14, wherein when the biological sample is incubated with the hyperbranched cationic polymer-modified silica membrane in the membrane-containing chamber (6) for selective DNA binding, a pressure of between about 0.01 bar to about 10 bar is applied to force the nucleic acid from the first layer (2) through to the second layer (3) of the chip (1) and eluted out.

18. The method according to claim 14, wherein the ratio of biological sample volume (SV) to wash buffer volume (BV) is 1:1 to about 1:8.

19. (canceled)

20. The method according to claim 14, wherein the elution buffer is applied to the elution buffer inlet port (5) between 1 to 4 times per extraction.

21. The method according to claim 14, wherein the extracted nucleic acid has a purity of greater than 90%.

22. The method according to claim 14, wherein the nucleic acid is a polymeric molecule, incorporating units of ribonucleic acid (RNA) or an analog thereof, or deoxyribonucleic acid (DNA) or an analog thereof.

23. The method according to claim 14, wherein the biological sample is selected from blood, blood plasma, blood components, saliva, urine, cerebrospinal fluid, urine, neonatal fluid, and aspirate.

24. (canceled)

25. A method of modifying the silica membrane in the membrane-containing chamber (6) of a microfluidic chip (1) comprising a membrane-containing chamber (6) bonded between a first layer (2) and a second layer (3), wherein the first layer (2) comprises a sample inlet port (4) in fluid communication with the membrane-containing chamber (6) by a first microchannel (8), and a wash buffer inlet port (10) and an elution buffer waste collection port (12) in fluid communication with the membrane-containing chamber (6) by a second microchannel (14); wherein the second layer (3) comprises an elution buffer inlet port (5) in fluid communication with an eluate collection port (7) adapted to deliver extracted nucleic acid to a nucleic acid amplification module via a third microchannel (9); and wherein the membrane-containing chamber (6) is in fluid communication with the second layer (3) through an extraction microchannel (11); and wherein the membrane-containing chamber (6) contains a silica membrane modified with a hyperbranched cationic group, the method comprising the steps of oxidising the silica membrane surface by applying an ultraviolet light to the surface; and curing the ultraviolet oxidised surface using a silanizing agent in an HPAE-solution to form the hyperbranched cationic polymer-modified surface.

26. (canceled)

27. The method of claim 25, wherein the curing step further comprises incubating the silica membrane with acetic acid.

28. (canceled)

29. The method of claim 25, wherein the HPAE solution is at a concentration of between 1 mg/ml and 50 mg/mL.

30. The method of claim 25, wherein the cationic group is selected from an amino ester, sulphonium, oxonium, phosphonium, imidazolium, pyrrolidinium, pyridinium, pipyridinium, morpholinium, and quinolinium.

31-34. (canceled)
